(19) United States
(12) Patent Application Publication
(10) Pub. No.: US 2023/0402175 A1
(43) Pub. Date: Dec. 14, 2023

(54) SYSTEM AND METHOD TO DELIVER EMERGENCY MEDICAL SERVICES DIRECTLY TO HEALTH CARE CONSUMERS

(71) Applicant: Wanda E. Cruz, Tampa, FL (US)

(72) Inventor: Wanda E. Cruz, Tampa, FL (US)

(21) Appl. No.: 18/036,463

(22) PCT Filed: Nov. 13, 2021
(86) PCT No.: PCT/US21/59276

§ 371 Date: May 11, 2023

Related U.S. Application Data
(60) Provisional application No. 63/113,348, filed on Nov. 13, 2020.

(51) Int. Cl. G16H 40/67; G16H 40/20; G16H 50/30; G16H 80/00; G08G 1/00; G06Q 30/04; G06Q 40/08

(52) U.S. Cl. CPC G16H 40/67; G16H 40/20; G16H 50/30; G16H 80/00; G08G 1/202; G06Q 30/04; G06Q 40/08

(57) ABSTRACT

A system and method to provide emergency medical services is disclosed which combines a control center staffed by a variety of health care practitioners equipped with medical diagnostic, treatment, telemedicine/health, satellite, communication, computing, clean energy technologies and related patient services into one coherent control center. Emergency medical services are provided to healthcare patients on-site or at their point of need. Automated systems of patient care/treatment and service procedures are used to integrate data with insurance companies with electronic medical records.

### FIELD OF THE INVENTION

The system and method of the invention relates to a system and method to provide emergency medical services. More specifically, the system and method combines a control center staffed by a variety of health care practitioners including medical doctors, nurses and mid-level health care practitioners equipped with medical diagnostic, treatment, telemedicine/health, satellite, communication, computing, clean energy technologies and related patient services such as prescription medication, insurance billing and healthcare follow up procedures into one coherent control center that provides emergency medical services to healthcare patients on-site or at their point of need. The control center of the system and method uses automated systems of patient care/treatment and service procedures including patient registration; use of triage data integration to produce patient acuity levels; and insurance and co-payment verification through integration of data with insurance companies with electronic medical records.

### BACKGROUND

Providing emergency medical care to patients in their home on demand in America is not realistically available today. In almost all cases, patients travel to emergency rooms or urgent care centers or are transported using ambulances or other emergency medical transport vehicles. The medical needs of the patient can be addressed by highly trained medical professionals to mid-level medical professionals, depending on the level of acuity of the patient.

There is a shortage of certain types of trained medical professionals in America, and there are professionals with varied levels of training and responsibility that can provide care.

A general practitioner or GP is a medical practitioner who provides primary care and specializes in nothing and sees everything. A general practitioner treats acute and chronic illnesses and provides preventive care and health education for all ages and both sexes. They have particular skills in treating people with multiple health issues and comorbidities.

There is currently a shortage of primary care physicians and also other primary care providers due to several factors. MLP (mid-level practitioners) trained health care providers who have a defined scope of practice. This means that they are trained and legally permitted to provide healthcare in fewer situations than physicians but more than other health professionals. For example, a mid-level provider may be trained for and legally permitted to perform some surgical procedures. They have highly variable levels of education and may have a formal credential and accreditation through the licensing bodies in their jurisdictions

A clinical nurse specialist (CNS) is an advanced practice nurse, with graduate preparation (earned master's or doctorate) from a program that prepares CNSs. CNSs are clinical experts in the diagnosis and treatment of illness, and the delivery of evidence-based nursing interventions. CNSs work with other nurses to advance their nursing practices and improve outcomes and provide clinical expertise to effect system-wide changes to improve programs of care.

A nurse practitioner (NP) is a registered nurse who has completed specific advanced nursing education (generally a master's degree or doctoral degree) and training in the diagnosis and management of common as well as a few complex medical conditions.

Nurse practitioners provide a broad range of healthcare services. Nurse practitioners treat both physical and mental conditions through comprehensive history taking, physical exams, physical therapy, ordering tests and therapies for patients, within their scope of practice. NPs can serve as a patient's “point of entry” health care provider and see patients of all ages depending on their designated scope of practice.

NPs prescribe physical therapy and other rehabilitation treatments including: prescribing drugs for acute and chronic illness (extent of prescriptive authority varies by state regulations); providing prenatal care and family planning services providing well-child care, including screening and immunizations; providing primary and specialty care services, health-maintenance care for adults, including annual physicals; providing care for patients in acute and critical care settings; assisting in minor surgeries and procedures (with additional training and usually under supervision) (e.g., emergent procedures, dermatological biopsies, suturing, casting); and counseling and educating patients on health behaviors, self-care skills, and treatment options

NPs practice in all U.S. states. The institutions in which they work may include, but are not limited to, the following: Community clinics, health centers, urgent care centers, emergency departments, etc. To be licensed as a nurse practitioner, the candidate must first complete the education and training necessary to be a registered nurse (RN).

A Registered Nurse (RN) is a health care professional responsible for implementing the practice of nursing through the use of the nursing process in conjunction with other health care professionals. Registered Nurses work as patient advocates for the care and recovery of the sick and maintenance of their health. In their work as advocates for the patient, RNs use the nursing process to assess, plan, implement, and evaluate nursing care of the sick and injured. RN's have a significantly expanded scope of practice, education and clinical training compared to that of licensed practical nurses.

Other medical professionals can also participate in the healthcare platform, including but not limited to dentists; optometrists; chiropractors; podiatrists; and opticians.

### SUMMARY OF THE INVENTION

The invention relates to a system and method providing a healthcare platform to provide emergency medical services comprising an application/software based control center staffed by a variety of health care practitioners equipped to provide on demand medical diagnostic services and treatment on site medical services or at a central control center. In one embodiment, the system and method provide emergency medical services to healthcare patients on-site or at their point of need. The control center of the system and method uses automated systems of patient care/treatment and service procedures including patient registration; use of triage data integration to produce patient acuity levels; and insurance and co-payment verification through integration of data with insurance companies with electronic medical records.

### BRIEF DESCRIPTION OF DRAWINGS

These and other features of this invention will be more readily understood from the following detailed description of the various aspects of the invention taken in conjunction with the accompanying drawings in which:

FIG. 1 depicts a flowscheme for automated patient registration according to one embodiment of the invention;

FIG. 2 depicts a flowscheme for automated doctor service selection according to one embodiment of the invention;

FIG. 3 depicts a flowscheme for automated video conferencing with patients according to one embodiment of the invention; and

FIGS. 4 and 5 depict flowschemes for automated management of shift changes of personnel according to one embodiment of the invention.

### DETAILED DESCRIPTION OF THE INVENTION

Illustrative embodiments will now be described more fully herein with reference to the accompanying drawings, in which embodiments are shown. The teachings may be practiced within any type of networked computing environment and is not limited by any embodiments. These embodiments are provided to convey the scope of the disclosure to those skilled in the art. In the description, details of well-known features and techniques may be omitted.

The invention relates to a system and method providing a healthcare platform to provide emergency medical services comprising an application/software based control center staffed by a variety of health care practitioners including medical doctors, nurses and mid-level health care practitioners equipped to provide on demand medical diagnostic services and treatment on site medical services or at a central control center, via telemedicine/health, satellite, communication, computing, clean energy technologies and related patient services such as prescription medication, insurance billing and healthcare follow up procedures. In one embodiment, the system and method provide emergency medical services to healthcare patients on-site or at their point of need. The control center of the system and method uses automated systems of patient care/treatment and service procedures including patient registration; use of triage data integration to produce patient acuity levels; and insurance and co-payment verification through integration of data with insurance companies with electronic medical records.

The control center comprises one or more application/software-based communication systems; record-keeping systems; computer systems; reference materials, libraries, and systems; medical or diagnostic kits, equipment, devices, or instruments; prescription and non-prescription drugs; means to write or fill a prescription; and one or more communication systems for contacting and communicating with a dispatch system. The control center tracks the location, health status, and vital signs of patients, and maintains a calendar of events for each patient, for example, appointments, device maintenance, administration of medication, and discharge.

The system and method allow a patient using digital devices on a communication system to access the control center to request emergency healthcare services. In one embodiment, the communication system comprises written, audio, visual, monitoring, or audio/visual communication, or a combination thereof. In one embodiment, the communication system comprises the Internet.

One embodiment of the healthcare platform enables a patient-initiated visit to address a healthcare issue with an issue-focused adaptive interview. The triage data resulting from this adaptive interview may be used to deploy the appropriate mobile medical vehicles to the point where the service was requested via an integrated end user GPS tracking location services on the patient's mobile device and a front line emergency medicine provider is deployed to not only diagnose but treat and where necessary stabilize the patient or discharge from the mobile medical vehicle once as medically indicated. Triage data are forwarded to a skilled clinician for review, who then provides an assessment and a plan of action for the issue. The plan of action may include on site treatment and stabilization, transfer to the control center for further monitoring and further evaluation including but not limited to radiologic studies, further treatments, transfer to a higher level of care or discharge with specific instructions, a prescription, or a referral to a third-party medical provider for testing, consultation, or treatment. The plan of action may include setting an on-site appointment for the patient. The plan of action may be application/software based, which plan of action may include dispatching a mobile medical vehicle to the patient's location to provide emergency medical services, if the patient has been evaluated by a video-conference provider or other outsource referring service or provider.

In one embodiment, the healthcare platform may determine a plan of action based on pre-determined factors identified in the triage data. The plan of action may include setting an on-site appointment for the patient or dispatching a mobile medical vehicle to the location of the patient. In one embodiment, the patient selects which type of service they wish to have but a telehealth doc may refer a mobile medical vehicle if the patient is of higher acuity. The staffing of the on-site appointment and the dispatching of the mobile medical vehicle may be automatically determined according to pre-determined factors identified in the triage data.

In one embodiment, the patient's primary care provider may be contacted, who will participate in the determination of the plan of action. The primary care provider can provide relevant information regarding the medical status and social conditions characteristic of the patient.

The control center may provide for follow up procedures for the patient including tests, prescriptions and perhaps more healthcare professional visits.

The control center processes the patient's information, records the visit and outcomes in their electronic health record and bills the insurance company.

The mobile medical vehicles may be equipped with life-support equipment and may be staffed with a level of healthcare professional set forth in the plan of action, depending on the level of acuity of the patient.

According to one embodiment of the system and method:
a patient accesses the control center via the internet through a downloadable mobile device application/software interface or a call center and registers with the system;
the patient provides his/her personal, healthcare and insurance information to the system through the control center;
the patient is provided with a member account and a personal electronic health record;
the patient provides background information about his/her healthcare needs through an issue-focused adaptive interview accessed through the control center;
the patient selects the type of service(s) provided by the control center.

Triage data resulting from the adaptive interview are forwarded to a skilled clinician associated with the system for review, who then provides an assessment and a plan of action for the health issue. The plan of action may include one or more of deployment of moderate to high acuity mobile medical vehicles; deployment of low acuity mobile medical vehicles; a videoconferencing evaluation by a medical provider or a control center in-person evaluation by a medical provider who will directly diagnose and treat the patient based on best medical practice standards and who may admit or discharge patient with specific instructions; a prescription; or a referral to a third-party medical provider for further testing, consultation, or treatment; setting of a video consultation with the patient; setting of an on-site appointment for the patient; or dispatch of a mobile medical vehicle to the patient's location to provide emergency medical services.

Prior to a patient visit to the control center, the patient's insurance company is contacted and the control center bills the patient's insurance company and applicable co-payments are collected directly via the application interface of the control center.

The control center can automatically assign an available room for the on-site patient visit.

The mobile medical vehicles may be outfitted to include a health care area that comprises treatment, diagnostic and minor surgical equipment. The mobile medical vehicle may comprise a power source to provide power and air to the health care area through air and electric lines. The power source may comprise a generator that is coupled to a fuel supply such as, but not limited to, propane, natural gas, diesel fuel, ethanol, gasoline, or combinations thereof. The power source may be used to run electrical equipment within the portable emergency vehicle (e.g., medical equipment, air filtration system, air conditioning, heating, lighting, water pumps, etc.), to provide power to external connections.

One or more patient areas adapted to diagnose, treat and/or perform minor surgery or first aid on a patient may be included in the mobile medical vehicle. One or more of the patient areas may include medical and/or diagnostic equipment for addressing, treating, or diagnosing a wide range of conditions or injuries including, but not limited to, emergent and resuscitative medical treatments and procedures, minor operative procedures, defined as those not requiring general anesthesia or deep sedation, such as skin lesion biopsy, wound repair, abscess incision and drainage. One or more of the patient areas may further comprise typical supplies found within a general practitioner's office, such as sample collection equipment, pressure cuffs, thermometers, etc. as well as equipment found within an analysis laboratory, such as polymerase chain reaction machines, hot water baths, culturing equipment, test strips, medical workstation, and the like. Thus, medical personnel may be able to diagnose an ongoing infectious epidemic within a population without having to send samples away from the mobile medical vehicle. Patient beds may be typical of those found in medical offices.

In one embodiment of the invention, healthcare professionals can log into a system of the control center to see if he/she has been assigned to any patient visits. Once the patient visit begins, the healthcare professional can provide updated status on the patient visit to the system, for example indicate time of arrival, document medical evaluations and treatments in real time, and billing amount.

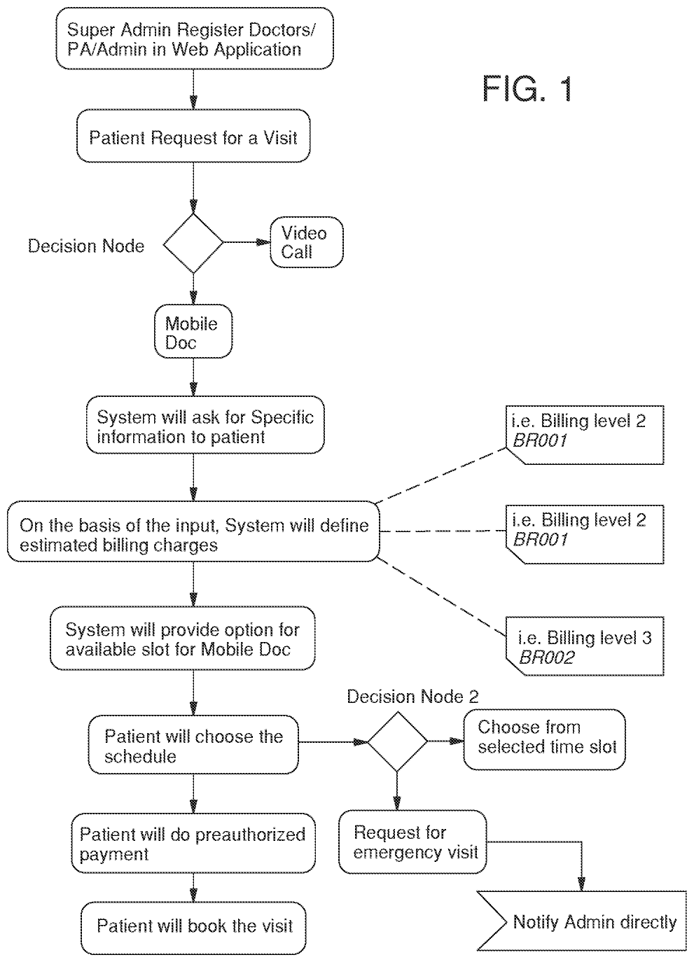

Turning to the figures, FIG. 1 depicts a flowscheme for automated patient registration according to one embodiment of the invention. At 100, a plurality of professionals and administrative personnel are registered with the system. At 105, a patient contacts the system to request assistance with a health issue and provides health information. At 110, the system automatically determines according to predetermined standards whether to have a video call with the patient at 115 or to deploy a mobile medical vehicle at 120. At 125, the system requests that the patient provide further specific information. At 130, the system will define estimated billing charges based on the specific information provided by the patient. The billing charges may for example be classified as Billing Level 2 or 3. At 135, the system will provide available time slots for the patient to be further assisted. At 140, the patient is provided with available options for further assistance. If a visit to the control center is selected, the patient selects a time slot at 145. If a mobile medical vehicle visit is selected, the patient requests an emergency visit at 150. If a mobile medical vehicle visit was selected at 150, the administrator of the system is directly notified at 155. Once the patient has selected an in-person visit or a mobile medical vehicle visit, the patient pre-authorizes payment at 160. At 165, once the patient has pre-authorizes payment, the patient visit is confirmed at 170.

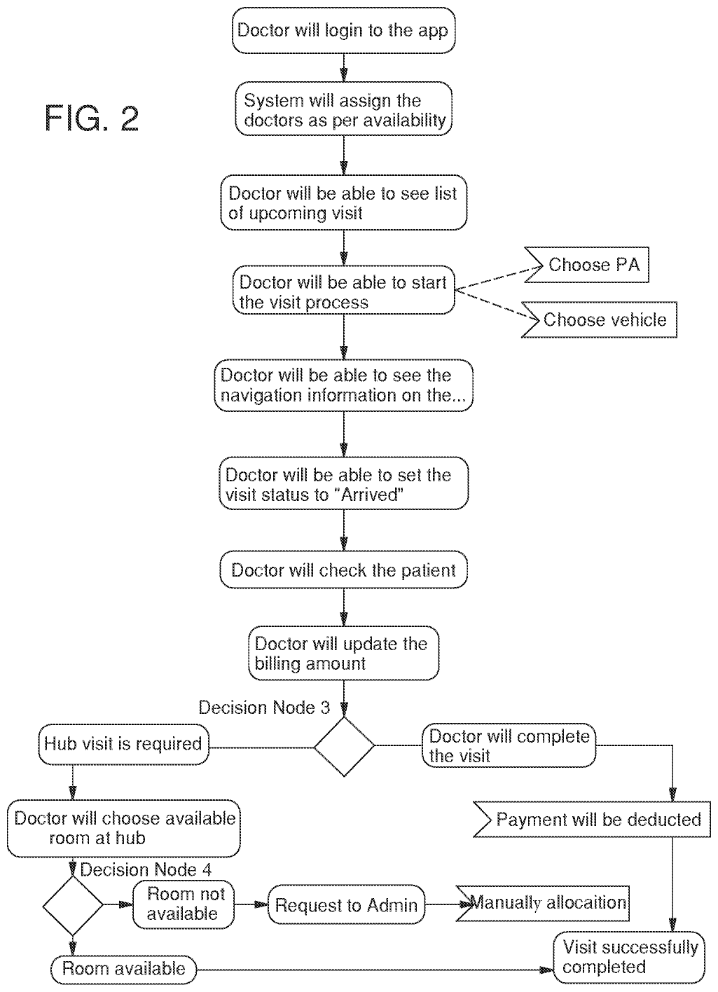

FIG. 2 depicts a flowscheme for automated doctor service selection according to one embodiment of the invention. At 200, a doctor who has previously registered with the system logs in. At 205, the system determines available appointments. At 210, the doctor is presented with the list of available appointments. At 215, the doctor selects a visit and starts a visit initiation process. At 220, the doctor selects a physician's assistant, if desired, and at 225, the doctor selects the level of mobile medical vehicle, if necessary. At 230, the doctor is presented with navigation information to the location of the patient. At 235, the doctor sets a visit status to indicate when she/he has arrived at the patient location. At 240, the doctor examines the patient. At 245, the doctor updates the billing charges, The doctor determines whether a visit to the control center is advised at 250 or instead whether she/he can complete the visit at the patient location at 255. If the control center visit is selected, the doctor will select an available room at 260. If a room is available at 265, the patient attends an in-person visit at the control center and completes the visit at 270. If a room is not available at 275, a request is sent to administration at 280 and at 285 a room is manually allocated. If the doctor completes the visit at 255, payment is completed at 290 and the visit is successfully completed at 270.

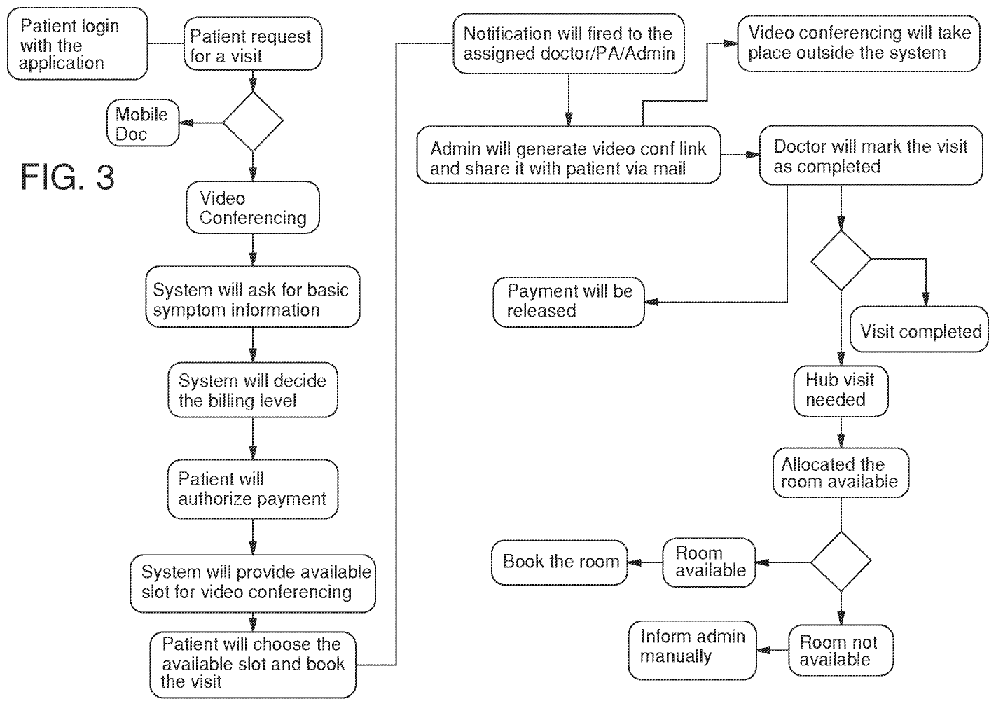

FIG. 3 depicts a flowscheme for automated video conferencing with a patient according to one embodiment of the invention. At 300, a patient logs into the system. At 305, the patient requests a visit. At 310, a visit with a mobile medical vehicle is requested at 315 or instead a video conference is requested at 320. If a video conference is requested, at 325, the system asks the patient for information on his/her symptoms. At 330, based on the symptoms, the system automatically determines a billing level. At 335, the patient authorizes payment. At 340, the system provides available time slots to hold a videoconference. At 345, the patient selects a time slot and books the visit. At 350, a notification is sent to the assigned doctor or other professional and administration of the booked video conference. At 355, administration of the system generates a video conference link and shares the link with the patient. At 360, the video conference takes place and at 365, the doctor or other professional may either mark the visit as completed at 368 in which case payment is released at 370. The doctor at 365 may determine that the patient should visit the control center at 375, in which case a room will be allocated for the visit at 380. If a room is available at 385, the room is booked at 390. If a room is not available at 395, administration is informed to manually book the room at 398.

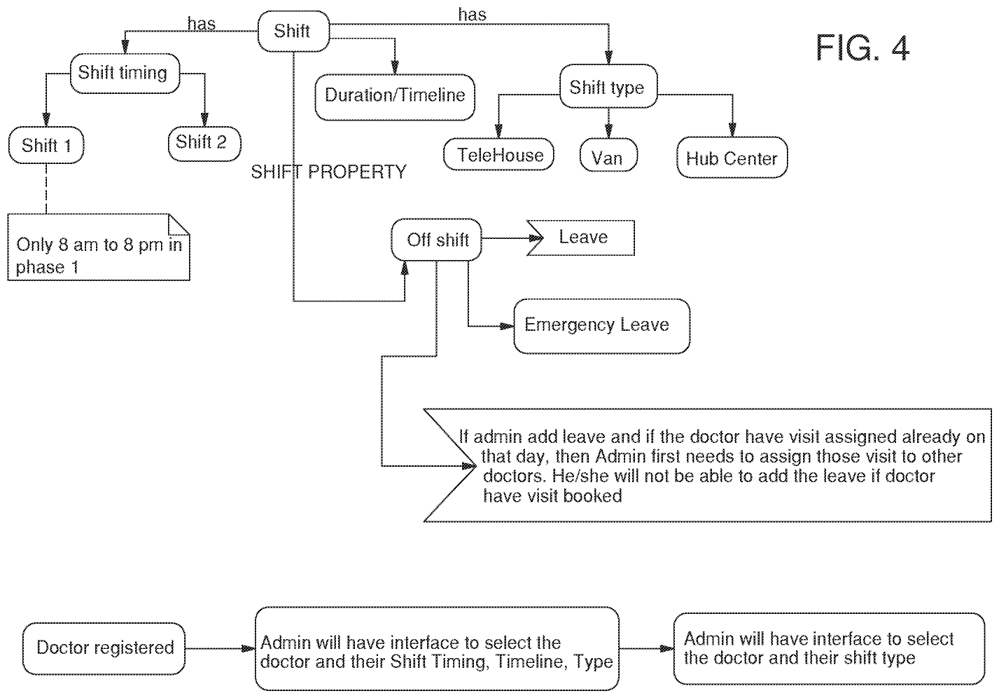

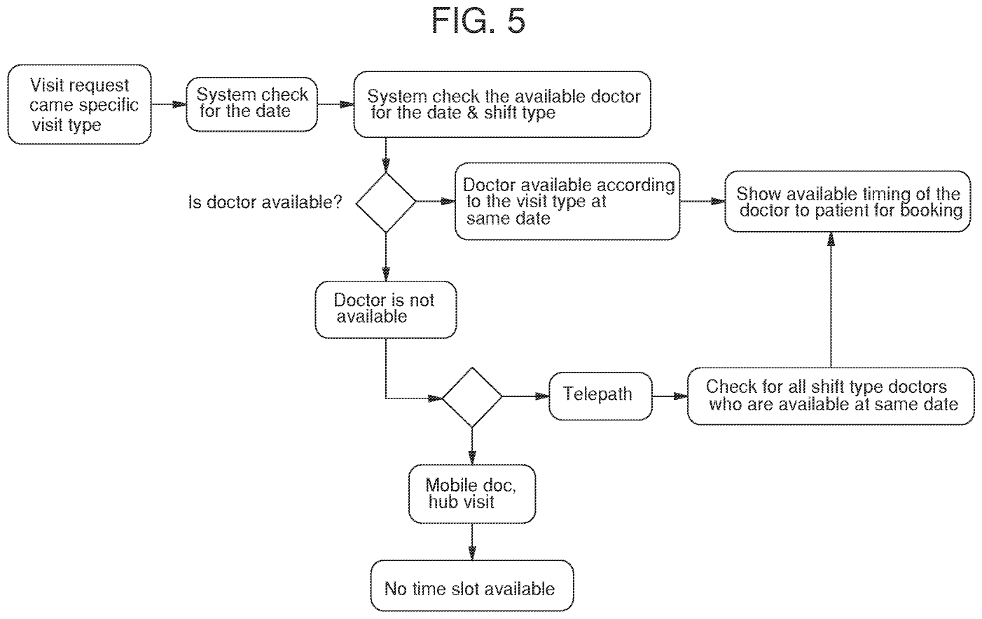

FIGS. 4 and 5 depict flowschemes for automated management of shift changes of personnel according to one embodiment of the invention.

At 400, a doctor has registered with the system. At 405 and 410, the administration of the system selects each registered doctor's shift timing, timeline and type of shirt. At 415 and 420, the system has predetermined shifts in duration and timeline. Each shift has a shift type at 425, which type may be video or teleconference 430; mobile medical vehicle 435; or control center 440. A registered doctor may be off-shift at 445 in which case the doctor can leave the control center at 435 or may decide to take emergency leave at 450. Any patient visits that had been assigned to a doctor who is going to be off-shift may be re-assigned to other doctors at 455. In one embodiment, a doctor who has been assigned to a patient visit cannot take leave at 460. At 465, each shift has a predetermined timing. In one embodiment, there may be 2 shifts per day, shift 1 at 470 and shift 2 at 475. In one embodiment, there may only be a single shift at 480, which for example may run from 8 a.m. to 8 p.m.

At 500, a patient may have made a request for a specific visit type. In this example, the patient has requested an in-person visit to the control center. At 505, the system will check for available personnel for that date. At 510, the system further checks for an available doctor or other professional at the requested time. If a doctor or other professional is available at 515, the system presents the doctor or other professional with the visit at 520 at which time the doctor or other professional can book the visit. If a doctor or other professional is not available at 525, the system will check for doctors or other professionals who are available for other types of visits at the requested time, in one instance, a teleconference or videoconference at 530. The system will check for available doctors or other professionals who can be booked for a teleconference or videoconference at 535. Any available doctors or other professionals who can book a teleconference or videoconference at the requested time will book it at 520. The system will also check for availability of a mobile medical vehicle at 540, which may not be available at 545 in which instance the patient will be requested to select a different time slot and/or visit type.

The foregoing description of various embodiments of the invention has been presented for purposes of illustration and description. It is not intended to be exhaustive or to limit the invention to the precise form disclosed and, obviously, many modifications and variations are possible. Such modifications and variations that may be apparent to a person skilled in the art are intended to be included within the scope of the invention as defined by the accompanying claims.

What is claimed is:

1. A healthcare platform for providing emergency medical services comprising:
a control center, the control center comprising a plurality of treatment rooms, medical diagnostic devices, medical treatment devices, telemedicine capabilities, video conferencing capabilities, communication capabilities, wherein the control center is operated via a central computer-based system; and
a plurality of mobile medical vehicles, wherein one or more of the plurality of mobile medical vehicles are dispatched automatically to locations remote from the control center by the central computer-based system according to one or more predetermined standards,
wherein a plurality of patients with a health issue in need of medical services provide information comprising triage data through an issue-focused adaptive interview to the central computer-based system, wherein further the central computer-based system is programmed to automatically determine an acuity level of one or more of the plurality of patients based upon the patient-provided information, wherein further an automatic dispatch of one or more of the mobile medical vehicles is determined according to the automatically determined patient acuity level.

2. The healthcare platform of claim 1, wherein one or more of the plurality of mobile medical vehicles are equipped with life-support equipment, wherein further the one or more of the plurality of mobile medical vehicles are staffed with one or more healthcare professionals, wherein the one or more healthcare professionals comprise one or more of a medical doctor, a registered nurse, a nurse practitioner, a physician's assistant, a dentist, an optometrist, a chiropractor, a podiatrist, an optician or a combination thereof, wherein a selection of the one or more healthcare professionals is commensurate with the patient acuity level.

3. The healthcare platform of claim 2, wherein one or more of the plurality of mobile medical vehicles is equipped to accommodate emergency treatment of patients according to the patient acuity level.

4. The healthcare platform of claim 1, wherein the triage data are forwarded to a healthcare professional, wherein the healthcare professional reviews the triage data and provides an assessment and a plan of action for the health issue of the patient.

5. The healthcare platform of claim 4, wherein the plan of action comprises control center treatment and stabilization, control center monitoring and evaluation, patient treatment at the control center, a teleconference with the patient, a video conference with the patient, transfer of the patient to a higher level of care, discharge of the patient with specific instructions, issuance of a prescription to the patient, a referral of the patient to a third-party medical provider, the setting of a control center appointment for the patient or the dispatch of a mobile medical vehicle to the location of the patient.

6. The healthcare platform of claim 1, wherein the central computer-based system determines an estimated billing charge based on the information provided by the patient.

7. The healthcare platform of claim 1, wherein the one or more patients contact the central computer-based system via a smart device or a telephone.

8. The healthcare platform of claim 1, wherein a plurality of healthcare professionals enroll with the central computer-based system to provide healthcare services to the one or more patients through the healthcare platform.

9. The healthcare platform of claim 8, wherein one or more of the plurality of healthcare professionals are automatically scheduled by the central computer-based system to provide services to one or more of the patients.

10. The healthcare platform of claim 1, wherein the patient provided information comprises insurance coverage information, wherein the central computer-based system confirms the insurance coverage information automatically with one or more insurance companies.

11. A computer-based healthcare method for providing emergency medical services to a patient comprising:
receiving information from a patient relating to a health issue;
conducting an issue-focused adaptive interview with the patient and receiving triage data in response;
automatically determining an acuity level of the patient based upon the triage data; and
dispatching one or more mobile medical vehicles to the location of the patient if the automatically determined patient acuity level meets predetermined standards.

12. The computer-based healthcare method of claim 11, wherein the triage data is automatically forwarded to one or more healthcare professionals, wherein the one or more healthcare professionals review the triage data and provide an assessment and a plan of action for the patient.

13. The computer-based healthcare method of claim 12, wherein the plan of action comprises treatment at a control center, monitoring and evaluation at the control center, patient treatment at the control center, a teleconference with the patient, a video conference with the patient, transfer of the patient to a higher level of care, discharge of the patient with specific instructions, issuance of a prescription to the patient, a referral of the patient to a third-party medical provider, the setting of a control center appointment for the patient or the dispatch of a mobile medical vehicle to the location of the patient.

14. The computer-based healthcare method of claim 12, wherein the one or more healthcare professionals comprise one or more of a medical doctor, a registered nurse, a nurse practitioner, a physician's assistant, a dentist, an optometrist, a chiropractor, a podiatrist, an optician or a combination thereof, wherein a selection of the one or more healthcare professionals is commensurate with the patient acuity level.

15. The computer-based healthcare method of claim 12, wherein one or more of the healthcare professionals are automatically scheduled to the patient.

16. The computer-based healthcare method of claim 11 further comprising automated estimation of estimated billing charges based on information provided by the patient.

17. The computer-based healthcare method of claim 11, wherein the patient contacts the computer-based healthcare method via a smart device or a telephone.

18. The computer-based healthcare method of claim 11, further comprising acquiring insurance coverage information from the patient and estimating billing charges based on the triage data.

19. The computer-based healthcare method of claim 18, further comprising automatically confirming the insurance coverage information with one or more insurance companies.
